(19) United States
(12) Patent Application Publication
(10) Pub. No.: US 2007/0224298 A1
(43) Pub. Date: Sep. 27, 2007

(54) INHIBITING 11(BETA)-HSD1 WITH CITRUS FLAVONOIDS

(76) Inventor: Shawn M. Talbott, Draper, UT (US)

Correspondence Address: HOLME ROBERTS & OWEN, LLP, 299 SOUTH MAIN, SUITE 1800, SALT LAKE CITY, UT, 84111, US

(21) Appl. No.: 11/690,683

(22) Filed: Mar. 23, 2007

Related U.S. Application Data
(60) Provisional application No. 60/785,160, filed on Mar. 23, 2006.

(51) Int. Cl. A61K 36/752; A61K 31/7048; A61K 31/353

(52) U.S. Cl. 424/736; 514/27; 514/456

(57) ABSTRACT

A therapeutically effective dose of polymethoxylated flavones, for example in the form of citrus peel extract, is provided for reducing the activity of 11(beta)-HSD1. Reducing the activity of 11(beta)-HSD1 in turn undesirable excess glucocorticoid, e.g. cortisol, levels. Particularly, the PMFs can be effectively used to inhibit a rise in glucocorticoids associated with the stress of weight loss. The polymethoxylated flavones may include one or more of tangeretin, nobiletin, and sinensetin. The PMFs may be administered in any of a variety of known modes of administrations such as oral and topical.

### CROSS-REFERENCE TO RELATED APPLICATIONS

This application claims the benefit of Provisional Application No. 60/785,160, filed Mar. 23, 2006, entitled “Inhibiting 11(beta)-HSD1 With Citrus Flavonoids,” which is incorporated by reference herein.

### BACKGROUND OF THE INVENTION

1. The Field of the Invention

The present invention relates to the fields of food compositions and dietary supplements. More particularly, the invention provides compositions and methods for the use of citrus peel extract for reducing the activity of the enzyme 11(beta)-HSD1.

2. The Relevant Technology

Obesity, diabetes, and metabolic syndrome (“Syndrome X”—encompassing insulin resistance, type 2 diabetes, dyslipidemia, hypertension, and abdominal obesity) are reaching epidemic proportions in the United States and around the world. Excess glucocorticoids such as cortisol (a primary stress hormone) produce visceral obesity and diabetes. 11(beta)-HSD1 is an enzyme that plays an important role in the cellular interconversion of glucorticoids between the active (cortisol) and inactive (cortisone) forms. Thus, 11(beta)-HSD1 is responsible for the modulation of cellular cortisol exposure and as such plays an important role in a number of common diseases associated with glucocorticoid excess including obesity, type 2 diabetes, age-related cognitive dysfunction, depression, osteoporosis, hypertension, arthritis, fibromyalgia, and dyslipidemias (hypercholesterolemia).

11(beta)-HSD1 is present in tissues of importance for metabolism and insulin sensitivity such as the liver and the adipose tissue. 11(beta)-HSD1 amplifies local glucocorticoid concentrations in these target tissues, even when circulating cortisol levels are low. In obese subjects, adipose levels of 11(beta)-HSD1 levels are markedly increased and animal models of elevated 11(beta)-HSD1 activity demonstrate visceral obesity, insulin resistance, diabetes, and dyslipidemia. Inhibition of 11(beta)-HSD1 in animals (pharmacologic and genetic) and humans (pharmacologic) results in enhanced insulin sensitivity.

The 11(beta)-HSD1 enzyme modulates tissue-specific glucocorticoid concentrations by generating active cortisol. Previous research has shown both liver 11(beta)-HSD1 and adipose tissue 11(beta)-HSD1 to be associated with hyperinsulinemia (diabetes) and visceral adiposity (abdominal obesity), respectively.

Rodent models of diabetes and obesity display elevated 11(beta)-HSD1 expression and transgenic mice with elevated 11(beta)-HSD1 develop visceral obesity, diabetes, and elevated cholesterol levels. Transgenic “knockout” mice (which lack 11(beta)-HSD1) are resistant to diabetes and obesity, even when fed a high-calorie diet. Obese and diabetic human subjects have been shown to exhibit elevated 11(beta)-HSD1 levels and activity—and inhibition of 11(beta)-HSD1 activity with selective or non-selective agents has been shown to improve insulin sensitivity.

A number of flavonoids are known to inhibit 11(beta)-HSD1, including those from grapefruit juice (naringenin), licorice (glycyrrhizin), soybeans (daidzein and genistein), apples (quercetin), and Chinese medicinal herbs (magnolia/magnolol, Perillae frutescens, Zizyphus vulgaris, and Scutellaria baicalensis).

Additional and improved methods for reducing 11(beta)-HSD1 would represent an advance in the art.

### BRIEF SUMMARY OF THE INVENTION

According to the invention, conditions enhanced by the presence of excess glucocorticoids can be reduced by reducing the activity of 11(beta)-HSD1 through the use of polymethoxylated flavones (PMFs), preferably in the form of citrus peel extract. For example, the PMFs can be effectively used to inhibit a rise in glucocorticoids associated with ongoing weight loss by reducing the activity of 11(beta)-HSD. The PMFs may be administered in any of a variety of known modes of administrations such as oral and topical.

Accordingly, a first example embodiment of the invention is a composition of matter that includes an effective dose of PMFs or citrus peel extract for reducing the activity of 11(beta)-HSD1. Another example embodiment of the invention is a method that includes administering to a mammal in need thereof a therapeutically effective dose of PMFs or citrus peel extract for reducing the activity of 11(beta)-HSD1, preferably including at least about 10 mg of the PMFs. The extract may be administered daily for a period such as at least two weeks to improve the effect.

The PMFs or citrus peel extract may be used in an oral dosage form for administration, for example as a beverage, a tablet, a capsule, a powder, a confectionary, a supplemented food, a liquid shake, or in a powdered mix having one or more additional ingredients suitable for forming a shake when mixed with a liquid. The citrus peel extract can also be administered topically.

Another embodiment of the invention is a packaged supplement for treating conditions associated with glucocorticoid excess. The packaged supplement may include: (a) a container which holds a plurality of effective doses of PMFs or citrus peel extract for reducing the activity of 11(beta)-HSD1; and (b) instructions for using the doses.

Another example embodiment of the invention is a method for promoting weight loss in a mammal undergoing weight loss. This example method generally includes: administering to a mammal participating in a weight loss exercise routine a diet consisting of a caloric intake that is insufficient to maintain the mammal's weight, and administering to the mammal a supplement as defined herein.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

In the following description, numerous specific details are set forth in order to provide a thorough understanding of the present invention. It will be obvious, however, to one skilled in the art that the present invention may be practiced without these specific details. In other instances, well-known aspects of citrus peel extract, polymethoxylated flavones (PMFs), and various oral and topical dosage forms have not been described in particular detail in order to avoid unnecessarily obscuring the present invention.

Polymethoxylated flavones, e.g. tangeritin, sinensetin, and nobilitin, are compounds that are extracted from citrus peels and are known to have health benefits in reducing cholesterol and promoting cardiovascular health. Without being limited by any particular theory, the mechanism by which PMFs exert their hypocholesterolemic effect is theorized to be by increased breakdown of the HMG-CoA Reductase enzyme.

According to the present invention, methods and compositions using the PMFs in citrus peel extract have been further shown to aid in weight loss and the treatment of diabetes and obesity. Thus, individuals at high risk of developing or having diabetes or desiring to prevent weight gain or promote weight loss may be treated with an effective dose of citrus peel extract or polmethoxylated flavones (PMFs). More particularly, the present invention relates to the discovery and use of PMFs for inhibiting the activity of 11(beta)-hydroxysteroid dehydrogenase-1(11(beta)-HSD1), reducing systemic and local cortisol concentrations (liver and adipose tissue), and promoting blood sugar control and weight loss.

With obesity, diabetes, and metabolic syndrome (“Syndrome X”—encompassing insulin resistance, type 2 diabetes, dyslipidemia, hypertension, and abdominal obesity) are reaching epidemic proportions in the United States and around the world, the current invention represents an important and innovative treatment for obesity, diabetes, and related metabolic diseases.

According to one embodiment of the invention, a mammalian subject can be administered once a day a composition of matter having from about 1 mg to about 1,000 mg of a citrus peel extract. Of course, the amount of citrus peel extract will preferably increase or decrease depending on the concentration of the citrus peel extract. In a more preferred embodiment the composition of matter has from about 100 mg to about 500 mg of a citrus peel extract. In a still more preferred embodiment the composition of matter at least about 300 mg of the citrus peel extract. In each of the foregoing example, the citrus peel extract preferably has at least about 10 mg PMFs, more preferably at least about 80 mg PMFs.

Alternatively, in another embodiment of the invention a mammalian subject can be administered once a day a composition of matter having one or more of tangeretin, nobiletin, and sinensetin. By way of example only, the dosage may have one or more of from about 1 mg to about 50 mg of tangeretin, from about 1 mg to about 50 mg of nobiletin, and from about 1 mg to about 20 mg of sinensetin. Of course, the dosage will increase or decrease depending on the concentration of the citrus peel extract. In a more preferred embodiment the composition of matter has from about 5 mg to about 20 mg of tangeretin, from about 5 mg to about 20 mg of nobiletin, and from about 2 mg to about 10 mg of sinensetin.

As used herein the term “an effective dose” indicates an amount sufficient to create a desirable result. For example, an effective dose can be the amount necessary to obtain a measurable change in blood sugar levels. An effective dose could therefore preferably reduce blood sugar levels level by at least 5 percent. Alternatively, an effective dose can be the amount necessary to promote a relative weight reduction wherein a relative weight loss is defined by a human obtaining a lower weight than the human user would have obtained without receiving the effective dosage. The precise parameters of an effective dose may of course vary with a number of factors, including by way of example only weight, gender, age, and desired result.

A variety of suitable forms of providing for usage of the disclosed compositions can be used and provided. General examples include nutritional supplements, pharmaceutical preparations, foods supplemented with the specified compositions of the invention, injections, and topical treatments such as suitably formed creams, ointments, and lotions. Such packaging and administration forms for the compositions are well known and are not discussed in detail herein to avoid obscuring the invention.

For example, such administration forms may include, by way of example only: tablets, capsules, injections, topical creams, ointments, lotions, dietary supplement in either solid or liquid form, and a food additive. For example, an effective dose of citrus peel extract can be combined in a diet shake. A diet shake can be provided to a user in either liquid form or as a powder having suitable ingredients to form a shake upon mixture with a liquid. In addition, a supplemented food such as a nutritional bar, dessert, or confectionary can be formed.

In addition, the citrus extract can be preferably combined with a variety of other supplements, vitamins, stabilizers, and the like as are known in the art to create a desirable administration form. By way of example only, such additives can include other forms of ginseng, caffeine, guarana, green tea extract, chromium, vanadium, CLA, and the like.

Where the dosage form is oral, a flavoring such as a citrus flavoring can be added to improve the taste as well as to enhance the user's perception of consuming the citrus peel extract. Where the dosage form is topical, a scent such as a citrus aroma can be added to not only improve the scent of the topical cream or ointment, but also to enhance the user's awareness of using the citrus peel extract.

Regardless of the dosage form, other conventional additives such as stabilizers, pH adjusters, excipients, carriers and diluents and the like may be added as is known in the art provided they do not interfere with the activity of citrus peel extract.

The compositions of the invention can be formulated so as to provide quick, sustained or delayed release of the citrus peel extract after administration employing procedures and formulations known in the art.

The following example is shown by way of illustration only.

### EXAMPLES

### Example 1

A group of fifty moderately overweight human subjects, self-described as “stress-eaters,” were selected and began an eight week study. The subjects had an average age of 44, an average starting weight of 78.5 kg, an average starting body fat percent of 32.4%, and an average starting waist circumference of 86.7 cm. During the eight week study, the subjects followed a program that included: daily supplements including citrus peel extract, a moderate calorie-restricted diet based on resting metabolic rate (RMR), a moderate exercise program on five days each week, and daily stress management techniques. More particularly, polymethoxylated Flavones (PMFs) were administered to the test subjects in the form of 300 mg of citrus peel extract/day containing 81 mg of PMFs comprising a minimum of 12 mg tangeretin, 12 mg nobiletin, and 5 mg sinensetin.

At the conclusion of the study the human subjects observed reduced body weight by 0.6 kg, reduced body fat by 5.0%, reduced fat mass by 1.9 kg, increased lean mass by 1.3 kg, and reduced waist circumference by 1.3 cm.

In addition, the subjects were tested before and after the eight week study for cortisol levels by salivary enzyme immunoassay and mood states by a Profile of Mood States. Results showed a drop in cortisol levels, an improved Global Mood State (MOOD as determined using a 65 question Profile of Mood States exam. See e.g. McNair D M, Lorr M, and Droppleman L F, Profile of Mood States Manual. San Diego, Calif.: Educational and Industrial Testing Services, 1971), and improved individual mood states as derived from the Profile of Mood States exam, as noted in Table 1 below.

TABLE 1

| Test | Percent Change |
|---|---|
| Salivary Cortisol | −18.7% |
| Global Mood State | −19.7% |
| Tension | −19% |
| Fatigue | −41% |
| Confusion | −14% |
| Vigor | +29% |
| Depression | −40% |
| Anger | −41% |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for preventing or treating conditions enhanced by the presence of excess glucocorticoids, comprising:
administering to a mammal in need thereof a therapeutically effective dose of citrus peel extract for reducing the activity of the enzyme 11(beta)-HSD1, the citrus peel extract comprising at least about 10 mg of polymethoxylated flavones (PMFs).

2. The method of claim 1, further comprising:
administering to a mammal the effective dose of citrus peel extract for reducing the activity of 11(beta)-HSD1 at least once a day for a period of at least two weeks.

3. The method of claim 1, wherein the therapeutically effective dose of citrus peel extract comprises from about 100 mg to about 500 mg.

4. The method of claim 1, wherein the citrus peel extract comprises:
from about 5 mg to about 20 mg tangeretin;
from about 5 mg to about 20 mg nobiletin; and
from about 2 mg to about 10 mg sinensetin.

5. The method of claim 1, wherein administering to a mammal an effective dose of citrus peel extract further comprises placing the citrus peel extract in an oral dosage form for administration, the oral dosage form comprising a liquid shake or a powdered mix having one or more additional ingredients suitable for forming a shake when mixed with a liquid.

6. The method of claim 1, wherein administering to a mammal an effective dose of citrus peel extract further comprises placing the citrus peel extract in an oral dosage form for administration, the oral dosage form selected from the group consisting of a beverage, a tablet, a capsule, a powder, a confectionary, and a supplemented food.

7. The method of claim 1, wherein administering to a mammal an effective dose of citrus peel extract further comprises placing the citrus peel extract in a topical dosage form for topical administration.

8. A composition of matter, comprising:
an effective dose of polymethoxylated flavones (PMFs) for reducing the activity of 11(beta)-HSD1.

9. The composition of matter of claim 8, wherein the PMFs comprise at least about 10 mg PMFs.

10. The composition of matter of claim 8, wherein the PMFs comprise at least about 80 mg polymethoxylated flavones (PMFs).

11. The composition of matter of claim 8, wherein the PMFs comprise at least one of tangeretin, nobiletin, and sinensetin.

12. The composition of matter of claim 11, wherein the PMFs comprise:
from about 5 mg to about 20 mg tangeretin;
from about 5 mg to about 20 mg nobiletin; and
from about 2 mg to about 10 mg sinensetin.

13. The composition of matter of claim 8, further comprising one or more additives selected from the group consisting of: ginseng, caffeine, guarana, green tea extract, chromium, vanadium, and CLA.

14. A packaged supplement for treating conditions associated with glucocorticoid excess, comprising: (a) a container which holds a plurality of the effective doses of PMFs for reducing the activity of 11(beta)-HSD1 as defined as in claim 8; and (b) instructions for using the doses.

15. The packaged supplement of claim 14, wherein each of the plurality of doses is separately packaged.

16. The packaged supplement of claim 14, wherein the plurality of doses comprise a single volume of liquid and the instructions for using the dietary supplement comprises instructions for metering and consuming a proper dose.

17. The packaged supplement of claim 14, wherein the dietary supplement comprises a topical treatment for administering the citrus peel extract transdermally.

18. A method for promoting weight loss in a mammal, the method comprising:
administering to a mammal participating in a weight loss exercise routine a diet consisting of a caloric intake that is insufficient to maintain the mammal's weight; and
administering to the mammal a composition of matter as defined as in claim 8.

19. A dietary supplement, comprising:
a dietary acceptable carrier prepared in a dosage form selected from the group consisting of a liquid, a tablet, a capsule, a powder, a confectionary, and a supplemented food; and
at least about 10 mg of polymethoxylated flavones.

20. A method for treating or ameliorating adverse symptoms in mammals associated with excess glucocorticoids, the method comprising administering daily for a plurality of days a dietary supplement as defined as in claim 19.
